(19) United States
(12) Patent Application Publication
(10) Pub. No.: US 2006/0234238 A1
(43) Pub. Date: Oct. 19, 2006

(54) Polymerase-based protocols for generating chimeric oligonucleotides

(76) Inventor: John C. Salerno, Averill Park, NY (US)

Correspondence Address: ELMORE PATENT LAW GROUP, PC, 209 MAIN STREET, N. CHELMSFORD, MA, 01863, US

(21) Appl. No.: 10/544,433

(22) PCT Filed: Feb. 6, 2004
(86) PCT No.: PCT/US04/03496

§ 371 Date: Nov. 28, 2005

Related U.S. Application Data
(60) Provisional application No. 60/445,689, filed on Feb. 6, 2003.
(60) Provisional application No. 60/445,703, filed on Feb. 6, 2003.
(60) Provisional application No. 60/446,045, filed on Feb. 6, 2003.
(60) Provisional application No. 60/445,704, filed on Feb. 6, 2003.
(60) Provisional application No. 60/474,063, filed on May 29, 2003.

(51) Int. Cl. C12Q 1/68; C12P 19/34

(52) U.S. Cl. 435/6; 435/91.2

(57) ABSTRACT

The present invention relates to methods of generating chimeric oligonucleotides without the need for subcloning. The methods of the invention are polymerase-based, and may optionally be adapted for use with reagents available in commercially available mutagenesis kits. Major applications of this method include directed evolution and other areas that benefit from the development of diversity.

### RELATED APPLICATIONS

This application claims the benefit of U.S. Provisional Application No. 60/445,704, filed on Feb. 6, 2003, U.S. Provisional Application No. 60/445,689, filed on Feb. 6, 2003, U.S. Provisional Application No. 60/445,703, filed on Feb. 6, 2003, U.S. Provisional Application No. 60/446,045, filed on Feb. 6, 2003, and U.S. Provisional Application No. 60/474,063, filed on May 29, 2003, Docket No. RPI-812, entitled “Parental Suppression via Polymerase-based Protocols for the Introduction of Deletions and Insertions.” The entire teachings of the above applications are incorporated herein by reference.

### BACKGROUND OF THE INVENTION

In recent years a number of methods have come into common use that allow the generation of site directed mutants without subcloning based on polymerase activity. This technology is mature enough to allow the sale of a number of mutagenesis kits that are capable of producing point mutants and in some case insertion and deletion mutants (‘indels’). Examples of such site-directed mutagenesis systems include Altered Sites® II in vitro Mutagenesis System (Promega Corporation, Madison, WI) and BD Transformer™ Site-Directed Mutagenesis Kit (BD BioSciences, Palo Alto, Calif.).

There are currently no comparatively quick, efficient and cost effective procedures for the production of chimeric oligonucleotides. Chimeric oligonucleotides are oligonucleotides that contain regions derived from two or more parent genes as opposed to site-directed mutagenized DNA comprising only point mutations or indels. Chimeric polynuceotides are useful in techniques such as “gene shuffling” (see, e.g. Crameri, A., et al., Nature 391 (6664):228-291 (1998)) and other processes aimed at producing proteins with novel properties.

It would be desirable to have quick, efficient and cost effective procedures for producing chimeric oligonucleotides that may optionally take advantage of existing commercially available kits and processes designed for site-directed mutagenesis or other purposes.

### SUMMARY OF THE INVENTION

The present invention relates to methods of generating chimeric oligonucleotides without the need for subcloning. The methods of the invention are polymerase-based, and may optionally be adapted for use with commercially available thermostable enzymes or with reagents available in commercially available mutagenesis kits such as Altered Sites® II in vitro Mutagenesis System (Promega Corporation, Madison, Wis.) and BD Transformer™ Site-Directed Mutagenesis Kit (BD BioSciences, Palo Alto, Calif.), to generate chimeric oligonucleotides in a quick, efficient and cost-effective manner.

In accordance with the invention, the first stage comprises: adding a forward primer to a cloning vector comprising a first target gene, wherein the 3′ end of the forward primer is complementary to a portion of the first target gene and the 5′ end of the forward primer is complementary to a region of a second target gene and synthesizing a first DNA strand comprising the forward primer by way of at least one cycle of single-primer linear amplification reaction, wherein the synthesis of the first DNA strand is halted prior to the 3′ end of the first target gene by a blocking oligonucleotide.

In accordance with the second stage of the invention, the first DNA strand produced in the first stage is mixed and hybridized with a cloning vector comprising a second target gene. The cloning vectors in the first and second stages of the method are identical except that each has a different target gene in their respective cloning sites. The first DNA strand functions as a primer for the second stage, single-primer linear amplification reaction. In the second stage single-primer linear amplification, the cloning vector comprising the second target gene is used as a template to extend the first DNA strand back around to the 5′ tail of the first DNA strand thereby copying the remaining vector including a region of the second target gene which is incorporated. The resulting product is a single-strand DNA (ssDNA) intermediate comprising the first and second target genes. The ssDNA intermediate is treated with a ligase to facilitate nick repair and recircularization of the ssDNA intermediate after each amplification reaction.

In the third stage, a reverse generic primer is added to the ssDNA intermediate produced in the second stage, and a single cycle of polymerase reaction produces the complementary reverse strand thereby forming a chimeric DNA duplex comprising first and second target genes. The chimeric DNA duplex can be used to transform competent or ultracompetent cells after optional nick repair via in vitro treatment with a ligase.

### BRIEF DESCRIPTION OF THE DRAWINGS

The foregoing and other objects, features and advantages of the invention will be apparent from the following more particular description of the preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a diagram outlining the basic method of the invention.

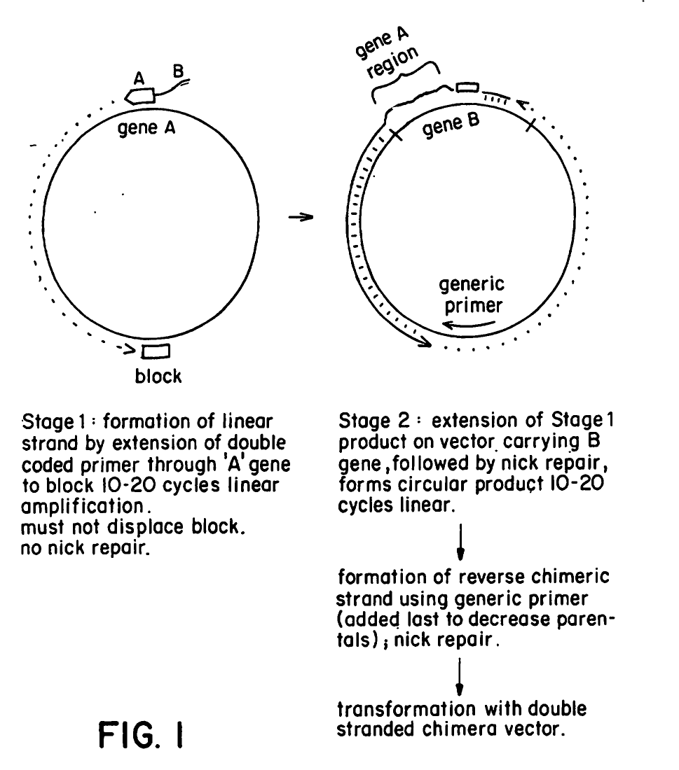

FIG. 2 is a diagram outlining an alternative embodiment of the invention.

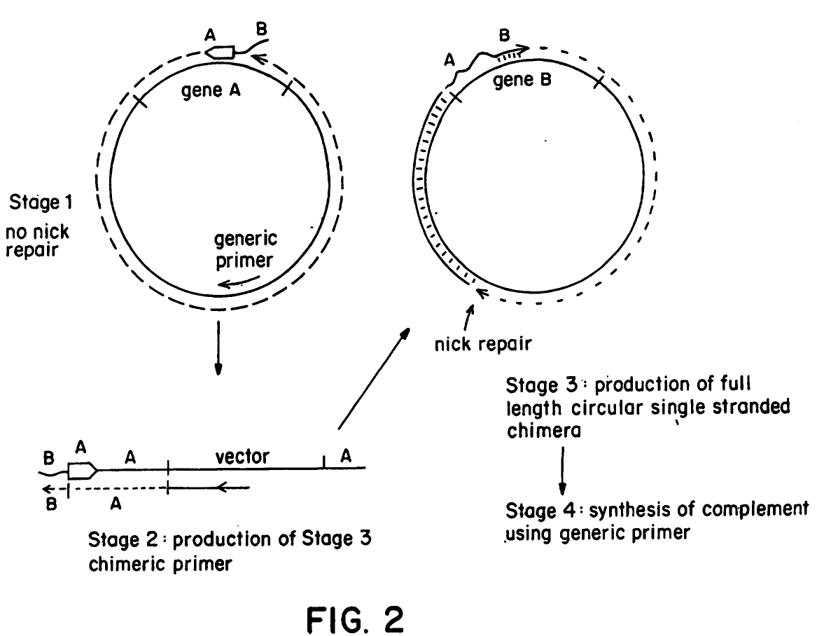

### DETAILED DESCRIPTION OF THE INVENTION

The present invention provides novel methods for the generation of chimeric oligonucleotides without the need for subcloning. In a first aspect, the method of the invention comprises the steps of:
(a) adding a forward primer and a first blocking oligonucleotide to a first parental DNA comprising a first target gene, wherein the forward primer comprises a 3′ region that is complementary to a region of the first target gene, and a 5′ tail that is complementary to a region of a second target gene;
(b) synthesizing by way of at least one cycle of a single-primer linear amplification reaction, a DNA strand comprising the forward primer and the first target gene, wherein the first blocking oligonucleotide is typically positioned to halt synthesis of the DNA strand prior to the 5′ end of the first target gene located in the first parental DNA;
(c) combining the first DNA strand with a second parental DNA comprising a second target gene, wherein the second parental DNA is identical to the first parental DNA except that each of the first and second parental DNAs comprise a different target gene in their respective cloning sites;
(d) synthesizing by means of at least one cycle of a single-primer linear amplification reaction, a single-stranded DNA intermediate (ssDNA intermediate) comprising regions derived from the first and second target genes, wherein the first DNA strand serves as a primer and the second parental DNA serves as a template for extending the first DNA strand around to the 5′ end of the first DNA strand;
(e) reacting a ligase with the ssDNA intermediate to repair nicks and to recircularize the ssDNA intermediate after each cycle of the single-primer linear amplification reaction carried out in step (d);
(f) combining a reverse generic primer with the ssDNA intermediate; and
(g) synthesizing by means of at least one cycle of primer extension, a reverse DNA strand that is complementary to the ssDNA intermediate thereby forming a chimeric DNA duplex comprising the first and second target genes.

The resulting chimeric DNA duplex can be used to transform competent or ultracompetent cells capable of expressing the chimera.

In one preferred embodiment, the method further comprises combining a ligase with the chimeric DNA duplex produced in step (g) to facilitate nick repair and recircularization of the chimeric DNA duplex prior to using the chimeric DNA duplex to transform host cells. In another embodiment, selection enzymes are used to digest the parental DNA strands after the synthesis steps. In yet another embodiment the chimeric DNA duplex can be subjected to optional PCR amplification, or linear amplification, such as about 10 or more cycles, to further increase the number of chimeric DNA product.

The terms “linear amplification reaction,” and “single-primer linear amplification reaction” as used herein, refer to a variety of enzyme mediated polynucleotide synthesis reactions that employ pairs of polynucleotide primers to linearly amplify a given polynucleotide and proceeds through one or more cycles, each cycle resulting in polynucleotide replication. A linear amplification reaction cycle typically comprises the steps of denaturing the double-stranded template, annealing the single primer or primers to the denatured template, and synthesizing polynucleotides from the primers. Thus the term “linear amplification reaction” as used herein is meant to include all of these steps. In the case of a single-primer linear amplification reaction, only one primer is used in each separate (meaning separate reaction vessels) single-primer linear amplification reaction. Linear amplification reactions used in the methods of the invention differ significantly from the polymerase chain reaction (PCR). The polymerase chain reaction produces an amplification product that grows exponentially in amount with respect to the number of cycles. Linear cyclic amplification reactions differ from PCR in that the amount of amplification product produced in a linear cyclic amplification reaction is linear with respect to the number of cycles performed. The reaction product accumulation rate laws differ because the products of each cycle in a PCR reaction are templates for the next cycle, while only the parentals are templates in a linear amplification. As in PCR linear amplification reaction cycle typically comprises the steps of denaturing the double-stranded template, annealing the single primer or primers to the denatured template, and synthesizing polynucleotides from the primers. The cycle may be repeated several times so as to produce the desired amount of newly synthesized polynucleotide product. Although linear amplification reactions differ significantly from PCR, guidance in performing the various steps of linear cyclic amplification reactions can be obtained from reviewing literature describing PCR and other polymerase based methods including, PCR: A Practical Approach, M. J. McPherson, et al., IRL Press (1991), PCR Protocols: A Guide to Methods and Applications, by Innis, et al., Academic Press (1990), and PCR Technology: Principals and Applications of DNA Amplification, H. A. Erlich, Stockton Press (1989). PCR is also described in many U.S. Patents, including U.S. Pat. Nos. 4,683,195, 4,683,202; 4,800,159; 4,965,188; 4,889,818; 5,075,216; 5,079,352; 5,104,792, 5,023,171; 5,091,310; and 5,066,584, which are hereby incorporated by references. Many variations of amplification techniques are known to the person of skill in the art of molecular biology. These variations include rapid amplification of DNA ends (RACE-PCR), amplification refectory mutation system (ARMS), PLCR (a combination of polymerase chain reaction and ligase chain reaction), ligase chain reaction (LCR), self-sustained sequence replication (SSR), Q-beta amplification, and strand displacement amplification (SDA), and the like. A person of ordinary skill in the art may use these methods to modify the linear amplification reactions used in the methods of the invention.

The term “first target gene” and “second target gene” as used herein refer to two different genes, gene regions or DNA sequences of interest, that are targeted for incorporation into the same chimeric oligonucleotide. In accordance with the invention, each of the first and second target genes is independently located within an identical cloning site in an identical vector. For convenience, the vector comprising the first target gene is referred to herein as the “first parental DNA” and the vector comprising the second target gene is referred to herein as the “second parental DNA”. In accordance with the invention, the first parental DNA serves as the template for the forward primer during the first stage of single primer linear amplification reaction to produce the DNA strand comprising the first target gene. In the second stage linear amplification reaction, the DNA strand produced in the first stage reaction functions as the primer and the second parental DNA serves as the template for extending the first DNA strand around to the 5′ end of the first DNA strand. It will be understood by one skilled in the art that each of the first target gene and second target gene could actually comprise more than one gene, gene region, or DNA sequence of interest, however, for convenience only, the invention will be described in terms of two target genes.

The term “forward primer” refers to the primer used in the first stage of the method of the invention that catalyzes the synthesis of a DNA strand that is complementary to the first target gene. The 3′ end of the forward primer comprises a region that is complementary to the first target gene and further comprises a 5′ tail that is complementary to the adjacent region of the second target gene. The primer extension catalyzed by the forward primer during the first stage single-primer linear amplification reaction is halted short of the 5′ end of the first target gene by an appropriate blocking oligonucleotide which is positioned to allow enough of the first parental DNA to be copied to produce significant overlap with the second parental DNA in the second stage linear amplification reaction.

The term “reverse generic primer” as used herein refers to a primer that anneals to the opposite strand of the second parental DNA as compared to the that of the forward primer and that is complementary to a region of the second parental DNA, wherein the region of complementarity does not overlap the region of the second parental DNA. Additionally, the generic reverse primer is preferably not complementary to any region of the forward primer.

The term “generic primer” as used herein refers to a primer that is complementary to any region of a parental strand that does not overlap the region of the parental strand targeted for mutation, and furthermore is not complementary to any other primer used in the reaction.

One skilled in the art will recognize that the either of the forward and reverse primers can be designed to be complementary to either the coding strand or the reverse strand of a target gene, gene sequence or other DNA of interest. The designation of forward and reverse primers in an association with a particular first or second reaction is for ease of discussion throughout and is not intended to be limiting.

In accordance with the invention, the first stage of the invention comprises combining the forward primer and a first blocking oligonucleotide with the first parental DNA and extending the forward primer by means of at least one cycle, preferably at least 10 cycles, even more preferably at least 20 cycles, of single-primer linear amplification reaction to the first blocking nucleotide to halt extension of the forward primer prior to the 5′ end of the first target gene (assuming for ease of discussion that the forward primer in the first reaction is homologous to the gene sequence). The first blocking oligonucleotide is complementary to any region downstream from the forward primer so long as the blocking oligonucleotide stops extension of the mutagenic primer prior to 5′ end of the first target gene but copies enough of the vector region to facilitate hybridization with the second parental DNA. The resulting product of the first stage of the method is referred to herein as the “first DNA strand”.

The second stage of the method comprises combining the first DNA strand produced in the first stage of the method with the second parental DNA. The first DNA strand serves as the primer and the second parental DNA serves as the template for extending the first DNA strand back around to the 5′ end of the first DNA strand by means of at least one cycle, preferably at least 10 cycles, even more preferably at least 20 cycles, of single primer linear amplification reaction thereby forming an excess of single-stranded DNA (ssDNA) intermediate. The ssDNA intermediate now comprises the first target gene and the second target gene as well as the vector region that is common to the first and second parental DNAs. A ligase is reacted with the ssDNA intermediate to for nick repair and recircularization of the ssDNA immediately after the synthesis phase of each cycle of linear amplification while the newly synthesized ssDNA is still hybridized with the closed second Parental DNA. Optionally, a selection enzyme is added to the second stage reactants to digest parental DNA after ligation.

The third stage of the method comprises combining a generic reverse primer with the ssDNA intermediate. The ssDNA intermediate serves as a template for extending (by way of one cycle of a primer extension reaction) the reverse generic primer to synthesize the complement of the ssDNA intermediate, thereby forming a chimeric DNA duplex comprising the first and second target genes. The transformation of competent or ultracompetent host cells with the DNA duplex yields a system capable of reproducing and expressing the chimera. In one preferred embodiment, a ligase is added to the third stage reaction for nick repair and recircularization prior to transformation.

In a second aspect of the invention, an alternative method of generating chimeric DNA is provided. The alternative method of the invention comprises the steps of:
(a) combining a forward primer with a first parental DNA comprising a first target gene, wherein the forward primer comprises a 3′ region that is complementary to a region of the first target gene and wherein the forward primer further comprises a 5′ tail that is complementary to a region of a second target gene;
(b) synthesizing by way of at least one cycle of a single-primer linear amplification reaction, a first DNA strand comprising the forward primer, wherein the first DNA strand is fully complementary to the first parental DNA;
(c) reacting the first DNA strand with a generic primer;
(d) extending the generic primer by means of at least one primer extension reaction to copy the first DNA strand thereby forming a chimeric primer comprising the complement of the first DNA strand;
(d) combining the chimeric primer of step (d) with a second parental DNA comprising a second target gene;
(e) extending by means of at least one cycle of single-primer linear amplification, the chimeric primer to copy the entire second parental DNA thereby forming a single-stranded DNA intermediate comprising the first target gene and the second target gene;
(f) reacting a ligase with the single-stranded DNA intermediate to repair nicks and circularize the single-stranded DNA intermediate after the synthesis phase of each cycle of the single-primer linear amplification carried out in step (e);
(g) combining a generic primer with the single-stranded DNA intermediate;
(h) synthesizing by means of at least one cycle of a single primer linear amplification reaction the complement to the single stranded DNA intermediate thereby forming a chimeric DNA duplex comprising the first and second target genes.

The resulting chimeric DNA duplex can be used to transform competent or ultracompetent cells capable of expressing the chimera. In one embodiment, a ligase is added after step (h) for nick repair of the chimeric DNA duplex prior to transforming a host cell with the chimeric DNA duplex. In another embodiment, selection enzymes may be used to digest the first and second parental strands after steps (d) and (e). Appropriate reaction conditions are maintained throughout the process of the invention to maximize desirable reaction products produced at each stage of the method of the invention while minimizing the production of artifact.

In accordance with the invention the host cells may be prokaryotic or eukaryotic. Preferably the host cells are prokaryotic, and preferably, the host cells for transformation are E. coli cells. In preferred embodiments, the cells are competent or ultracompetant cells. Ultracompetant cells such as the SL10-Gold® ultracompetetant cells available from Stratagene (La Jolla, Calif.) are particularly useful for the transformation of large DNA molecules with high efficiency.

Techniques for preparing and transforming competent single cell microorganisms are well know to the person of ordinary skill in the art and can be found, for example, in Sambrook et al., Molecular Cloning: A Laboratory Manual Coldspring Harbor Press, Coldspring Harbor, N.Y. (1989), Harwood Protocols For Gene Analysis, Methods In Molecular Biology Vol. 31, Humana Press, Totowa, N.J. (1994), and the like. Frozen competent cells may be transformed so as to make the methods of the invention particularly convenient.

The term “oligonucleotide” as used herein with respect to the primers and the blocking oligonucleotides is used broadly. Oligonucleotides include not only DNA but various analogs thereof. Such analogs may be base analogs and/or backbone analogs, e.g., phosphorothioates, phosphonates, and the like. Techniques for the synthesis of oligonucleotides, e.g., through phosphoramidite chemistry, are well known to the person ordinary skilled in the art and are described, among other places, in Oligonucleotides and Analogues: A Practical Approach, ed. Eckstein, IRL Press, Oxford (1992). Preferably, the oligonucleotide used in the methods of the invention are DNA molecules.

In accordance with the invention, parental DNA strands used as templates during linear amplification reactions may optionally be digested during the process of the invention. By performing the digestion step, the number of transformants containing non-chimeric oligonucleotides is reduced. The term “digestion” as used herein in reference to the enzymatic activity of a selection enzyme is used broadly to refer both to (i) enzymes that catalyze the conversion of a polynucleotide into polynucleotide precursor molecules and to (ii) enzymes capable of catalyzing the hydrolysis of at least one bond on polynucleotides so as to interfere adversely with the ability of a polynucleotide to replicate (autonomously or otherwise) or to interfere adversely with the ability of a polynucleotide to be transformed into a host cell. Restriction endonucleases are an example of an enzyme that can “digest” a polynucleotide. Typically, a restriction endonuclease that functions as a selection enzyme in a given situation will introduce a specific single cleavage into the phosphodiester backbone of the template strands that are digested.

The term “selection enzyme” refers to an enzyme capable of catalyzing the digestion of a polynucleotide template for mutagenesis. Examples of selection enzymes include restriction endonucleases. One suitable selection enzyme for use in the parental strand digestion step is the restriction endonuclease Dpn I, which cleaves the polynucleotide sequence GATC only when the adenine is methylated (6-methyl adenine). Suitable selection enzymes are provided with commercially available mutagenesis kits such as the QuikChange® Site Directed Mutageneisis System kit supplied by Stratagene (La Jolla, Calif.), Altered Sites® II in vitro Mutagenesis System (Promega Corporation, Madison, Wis.) and BD Transformer™ Site-Directed Mutagenesis Kit (BD BioSciences, Palo Alto, Calif.).

In alternative embodiments, alternate selection processes may be used. In these embodiments, each parental DNA comprises one “live” and one “dead” restriction site (assuming the respective target gene did not contain the same restriction sites) or alternatively one “live” and “one” dead antibiotic resistance sites. The first and second blocking oligonucleotides are designed such that each respective blocking oligonucleotide is located between the selection sites (either resistance or restriction). Only the chimera produced in accordance with the method of the invention will have either both antibiotic resistance sites or alternatively, neither of the restriction sites.

The primers of the invention are preferably about 20-50 bases in length, and more preferably about 25 to 45 bases in length. However, in certain embodiments of the invention, it may be necessary to use primers that are less than 20 bases or greater than 50 bases in length so as to obtain the mutagenesis result desired. The primers may be of the same or different lengths. In some embodiments, 5′ phosphorylation may be desirable. 5′ phosphorylation may be achieved by a number of methods well known to a person of ordinary skill in the art, e.g., T-4 polynucleotide kinase treatment. Preferably, the target gene region of the primers are flanked by about 10-15 bases of correct, i.e., non-mismatched, sequence so as to provide for the annealing of the primer to the template DNA strands for mutagenesis. In preferred embodiments of subject methods, the GC content of primers is at least 40%, so as to increase the stability of the annealed primers. Preferably, the primers are selected so as to terminate in one or more G or C bases. Very high GC content (over 70%), or runs of more than five successive GC bases, are not desirable since this decreases specificity.

In one embodiment of the invention, the invention may be carried out using the reagents provided in commercially available mutagenesis kits such as Altered Sites® II in vitro Mutagenesis System (Promega Corporation, Madison, WI) and BD Transformer™ Site-Directed Mutagenesis Kit (BD BioSciences, Palo Alto, Calif.). However, the present invention may be practiced without the use of a commercially available kit so long as a high fidelity, thermostable polymerase and high competence cells are used in the present method.

The single-primer linear cyclic amplification reaction may be catalyzed by a thermostable or non-thermostable high-fidelity polymerase. Polymerases for use in the linear cyclic amplification reactions of the subject methods have the property of not displacing the primers and blocking oligonucleotides that are annealed to the template. Preferably, the polymerase used is a thermostable polymerase. The polymerase used may be isolated from naturally occurring cells or may be produced by recombinant DNA technology. The use of Pfu DNA polymerase (Stratagene, La Jolla, Calif.), a DNA polymerase naturally produced by the thermophilic archae Pyrococcus furiosus, is one preferred enzyme for use in the linear cyclic amplification reaction steps of the claimed invention. Other high fidelity polymerases suitable for use in the present invention include, but are not limited to, KOD HiFi (EMD Biociences Inc, Madison, Wis.), MA), Phusion™ High-Fidelity Polymerase (Finnzymes, Espoo, Finland).

Synthesis of the new DNA strands takes place during the synthesis phase of the single-primer linear amplification reaction(s). However, prior to the synthesis phase of the reaction, the single-primer linear amplification reaction necessarily includes the steps of denaturing the double-stranded DNA followed by annealing of the primer that is present in the reaction as described earlier.

The single-primer linear amplification reactions as employed in the methods of the invention are preferably carried out to the limits imposed by the polymerase properties and the amplification conditions. Preferably the number of cycles in the linear cyclic amplification reaction step is at least 10 cycles, more preferably at least 20 cycles, and even more preferably at least 25 or more cycles. The limitation on the optimum cycle number is specific to the application and is imposed by runaway PCR artifact triggered by low probability nonspecific binding. This is a problem primarily in very GC rich regions, and can be overcome by decreasing the number of cycles and increasing the parental DNA template present in the reaction.

Another aspect of the invention is to provide kits for performing the methods of the invention. The kits of the invention provide one or more of the enzymes or other reagents for use in performing the subject methods. The kits may contain reagents in pre-measured amounts so as to ensure both precision and accuracy when performing the subject methods. Kits may also contain instructions for performing the methods of the invention. In one embodiment, kits of the invention comprise at least one polymerase, a ligase and instructions for carrying out the method. The kits may also comprise a DNA vector comprising a cloning site, ultracompetent cells and blocking oligonucleotides complementary to regions of the DNA vector. Kits of the invention may also comprise individual nucleotide triphosphates, mixtures of nucleoside triphosphates (including equimolar mixtures of DATP, dTTP, dCTP and dGTP), and concentrated reaction buffers. In a preferred embodiment, the kits comprise at least one DNA polymerase, concentrated reaction buffer, a nucleoside triphosphate mix of the four primary nucleoside triphosphates in equal amounts, frozen competent or ultracompetent cells and instructions for carrying out the method.

One skilled in the art will appreciate the many advantages that the method of the invention provides. For example, the improved site-directed mutagenesis methods of the invention are useful in protein and enzyme engineering technologies for the production of industrial proteins and enzymes such as detergent enzymes, enzymes useful for neutralizing contaminants and enzymes useful as fuel additives. Likewise, the methods of the invention are useful in protein engineering technologies for the production of proteins and enzymes useful and the food and life sciences industries such as primary and secondary metabolites useful in the production of antibiotics, proteins and enzymes for the food industry (bread, beer) and combinatorial arrays of proteins for useful in generating multiple epitopes for vaccine production.

The methods of the invention can also be used in the production of mutagenized fusion proteins. A DNA sequence targeted for mutagenesis is tagged or fused with the DNA sequence encoding a known protein (e.g. maltose binding protein (MBP) or green fluorescent protein (GFP)). For example, vectors with a GFP gene adjacent to a cloning site would allow easy conversion of a vector for expression of a target gene to one of several possible target gene-GFP mutants with different linkers. These in turn could be targeted to different cell locations by modifications of the opposite (usually N) terminus. Kits designed identify fusion proteins are advantageously used to identify and isolate the mutagenized protein of interest in vitro (western blots) or in tissue using anti-GFP.

The patent and scientific literature referred to herein establishes the knowledge that is available to those with skill in the art. All United States patents and published or unpublished United States patent applications cited herein are hereby incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of generating chimeric oligonucleotides comprising the steps of:
a) adding a forward primer and a first blocking oligonucleotide to a first parental DNA comprising a first target gene, wherein the forward primer comprises a 3′ region that is complementary to a region of the first target gene, and a 5′ tail that is complementary to a region of a second target gene;
(b) synthesizing by way of at least one cycle of a single-primer linear amplification reaction, a DNA strand comprising the forward primer and the first target gene, wherein the first blocking oligonucleotide is typically positioned to halt synthesis of the DNA strand prior to the 5′ end of the first target gene located in the first parental DNA;
(c) combining the first DNA strand with a second parental DNA comprising a second target gene, wherein the second parental DNA is identical to the first parental DNA except that each of the first and second parental DNAs comprise a different target gene in their respective cloning sites;
(d) synthesizing by means of at least one cycle of a single-primer linear amplification reaction, a single-stranded DNA intermediate (ssDNA intermediate) comprising regions derived from the first and second target genes, wherein the first DNA strand serves as a primer and the second parental DNA serves as a template for extending the first DNA strand around to the 5′ end of the first DNA strand;
(e) reacting a ligase with the ssDNA intermediate to repair nicks and to recircularize the ssDNA intermediate after each cycle of the single-primer linear amplification reaction carried out in step (d);
(f) combining a reverse generic primer with the ssDNA intermediate; and
(g) synthesizing by means of at least one cycle of primer extension, a reverse DNA strand that is complementary to the ssDNA intermediate thereby forming a chimeric DNA duplex comprising the first and second target genes.

2. The method of claim 1 further comprising transforming an ultracompetent host cell with the chimeric DNA duplex of step (f).

3. The method of claim 1 wherein the single primer linear amplification reaction of steps (c) and (d) are each repeated for at least 20 cycles.

4. The method of claim 1 wherein the primer extension reaction of step (g) is carried out for 1 cycle.

5. The method of claim 1 wherein the linear amplification reactions of steps (c), and (d) are catalyzed by pfu DNA polymerase.

6. The method of claim 1 wherein the primer extension reaction of step (f) is catalyzed by pfu DNA polymerase.

7. The method of claim 1 wherein the linear amplification reactions of steps (c) and (d) are catalyzed by a thermostable DNA polymerase.

8. The method of claim 1 further comprising reacting a ligase with the chimeric DNA duplex formed in step (f).

9. The method of claim 1 further comprising a digestion step subsequent to the synthesis of step (b) to reduce the amount of the first parental DNA in the reaction mixture.

10. The method of claim 1 further comprising a digestion step subsequent to the synthesis of step (d) to reduce the amount of the second parental DNA in the reaction mixture.

11. A kit for use in the method of claim 1 comprising, a DNA polymerase, a ligase and instructions for carrying out the method.

12. The kit of claim 11 further comprising competent or ultracompetent cells.

13. The kit of claim 11 further comprising a DNA vector comprising a cloning site and blocking oligonucleotides complementary to one or more regions of the DNA vector.

14. The kit of claim 11 further comprising concentrated buffers for carrying out the method of the invention.

15. The kit of claim 11 further comprising individual nucleotide triphosphates, or mixtures of nucleoside triphosphates.

16. A method of using a kit comprising a DNA polymerase, a ligase and instructions for carrying out the method, in a method comprising the steps of:
(a) adding a forward primer and a first blocking oligonucleotide to a first parental DNA comprising a first target gene, wherein the forward primer comprises a 3′ region that is complementary to a region of the first target gene, and a 5′ tail that is complementary to a region of a second target gene;
(b) synthesizing by way of at least one cycle of a single-primer linear amplification reaction, a DNA strand comprising the forward primer and the first target gene, wherein the first blocking oligonucleotide is typically positioned to halt synthesis of the DNA strand prior to the 5′ end of the first target gene located in the first parental DNA;
(c) combining the first DNA strand with a second parental DNA comprising a second target gene, wherein the second parental DNA is identical to the first parental DNA except that each of the first and second parental DNAs comprise a different target gene in their respective cloning sites;
(d) synthesizing by means of at least one cycle of a single-primer linear amplification reaction, a single-stranded DNA intermediate (ssDNA intermediate) comprising regions derived from the first and second target genes, wherein the first DNA strand serves as a primer and the second parental DNA serves as a template for extending the first DNA strand around to the 5′ end of the first DNA strand;
(e) reacting a ligase with the ssDNA intermediate to repair nicks and to recircularize the ssDNA intermediate after each cycle of the single-primer linear amplification reaction carried out in step (d);
(f) combining a reverse generic primer with the ssDNA intermediate; and
(g) synthesizing by means of at least one cycle of primer extension, a reverse DNA strand that is complementary to the ssDNA intermediate thereby forming a chimeric DNA duplex comprising the first and second target genes.

17. The method of claim 16 wherein the kit further comprises competent cells.

18. The method of claim 17 further comprising the step of transforming a host cell with the chimeric DNA duplex of step (g).

19. A method of generating chimeric DNA comprising the steps of:
(a) combining a forward primer with a first parental DNA comprising a first target gene, wherein the forward primer comprises a 3′ region that is complementary to a region of the first target gene and wherein the forward primer further comprises a 5′ tail that is complementary to a region of a second target gene;
(b) synthesizing by way of at least one cycle of a single-primer linear amplification reaction, a first DNA strand comprising the forward primer, wherein the first DNA strand is fully complementary to the first parental DNA;
(c) reacting the first DNA strand with a generic primer;
(d) extending the generic primer by means of at least one primer extension reaction to copy the first DNA strand thereby forming a chimeric primer comprising the complement of the first DNA strand;
(d) combining the chimeric primer of step (d) with a second parental DNA comprising a second target gene;
(e) extending by means of at least one cycle of single-primer linear amplification, the chimeric primer to copy the entire second parental DNA thereby forming a single-stranded DNA intermediate comprising the first target gene and the second target gene;
(f) reacting a ligase with the single-stranded DNA intermediate to repair nicks and circularize the single-stranded DNA intermediate after the synthesis phase of each cycle of the single-primer linear amplification carried out in step (e);
(g) combining a generic primer with the single-stranded DNA intermediate;
(h) synthesizing by means of at least one cycle of a single primer linear amplification reaction the complement to the single stranded DNA intermediate thereby forming a chimeric DNA duplex comprising the first and second target genes.

20. The method of claim 20 further comprising transforming a host cell with the chimeric DNA duplex of step (h).

21. A kit for use in the method of claim 19 comprising, a DNA polymerase, a ligase and instructions for carrying out the method.
